(19) United States
(12) Patent Application Publication
(10) Pub. No.: US 2025/0145979 A1
(43) Pub. Date: May 8, 2025

(54) PROTEIN HAVING GLUCOCEREBROSIDASE ACTIVITY AND METHOD FOR PRODUCING SAME

(71) Applicant: NIPPON SHOKUBAI CO., LTD., Osaka-shi, Osaka (JP)

(72) Inventors: Daisuke TATEIWA, Suita-shi, Osaka (JP); Yuki MAKINO, Suita-shi, Osaka (JP)

(21) Appl. No.: 18/833,882

(22) PCT Filed: Jan. 26, 2023
(86) PCT No.: PCT/JP2023/002410

§ 371 Date: Jul. 26, 2024

(30) Foreign Application Priority Data
Jan. 31, 2022 (JP) 2022-013052

(51) Int. Cl. C12N 9/24

(52) U.S. Cl. CPC C12N 9/2402; C12Y 302/01045

(57) ABSTRACT

The present invention provides a protein capable of preventing viral infection and having glucocerebrosidase activity. There is provided a protein, (a) containing an amino acid sequence set forth in SEQ ID NO: 1 or 2 or an amino acid sequence having identity of 90% or more therewith, (b) being added with no sugar chain, and (c) having glucocerebrosidase activity.

### TECHNICAL FIELD

The present invention relates to a protein having glucocerebrosidase activity and a method for producing the protein.

### BACKGROUND ART

Lysosomal disease is a hereditary disease caused by activity decrease or defects in lysosomal enzymes and their related factors and by the resultant storage of the substrates of such enzymes in the living body. For example, in Gaucher disease that is one type of lysosomal disease, activity decrease in glucocerebrosidase (β-glucosidase; GBA) causes storage of glucocerebroside in cells such as macrophages in reticuloendothelial tissues, and as a result, symptoms and observations such as splenohepatomegaly; anemia and decreased platelet count associated with enhancement of splenic function; bone lesions; increase in the levels of blood acidic phosphatase and angiotensin converting enzyme; and the like are observed (Non-Patent Literature 1).

As a method for treating such a lysosomal disease, enzyme replacement therapy has been frequently employed heretofore. For example, in Gaucher disease, a recombinant enzyme expressed by using a cDNA encoding human glucocerebrosidase in a Chinese hamster ovary (CHO) cell strain is used with a sugar chain-altered form so that to be uptaken into target cell macrophages easily (for example, mannose residues are added at the non-reducing terminal of the enzyme in order to facilitate recognition by the mannose receptor present on the surface of target cell macrophages).

However, production of a recombinant enzyme using a mammalian cultured cell such as the above-described CHO cell strain as a host has problems in that a culture solution is expensive, there is a risk of infection by a zoonotic virus, and proliferation of cells is slow. A technique for producing a recombinant of glucocerebrosidase (GBA) using a cell derived from a plant as a host has also been proposed (Patent Literature 1). However, in a recombinant enzyme produced using a plant or a Saccharomyces cerevisiae as a host among eukaryotic organisms, the structure of a sugar chain added to the enzyme by post-translational modification is greatly different from that of a mammalian cell, and thus there is a problem in that the recombinant enzyme exhibits antigenicity to a mammal. Therefore, there is a problem in that a recombinant enzyme produced using a cell derived from a wild-type plant or a Saccharomyces cerevisiae as a host is used as a biopharmaceutical drug.

Meanwhile, a technique for producing a recombinant glucocerebrosidase (GBA) using a cell derived from a prokaryote such as Escherichia coli as a host is also known. According to such a technique, problems of infection risk, productivity, and production cost as in the case of using a eukaryotic organism as a host as described above do not occur. Since the prokaryote does not undergo glycosylation, which is post-translational modification of the protein, there is no problem of antigenicity derived from the added sugar chain.

However, Non-Patent Literature 2 describes that the binding of a sugar chain to at least one glycosylation site (sugar chain binding site) is necessary for the formation of an active GBA protein. In addition, Patent Literature 1 describes that a recombinant GBA protein expressed in Escherichia coli does not have enzyme activity. As described above, it is a common recognition of those skilled in the art that the recombinant GBA protein produced by a prokaryote does not have a desired activity because a sugar chain is not added by post-translational modification.

### CITATION LIST

### Patent Literature

Patent Literature 1: JP 2006-524506 A

### Non-Patent Literature

Non-Patent Literature 1: Bou-Gharios G. et al., Histochem J. (1993) 25, 593-605
Non-Patent Literature 2: Marie E. et al., Biochem. Biophys. Res. Commun. (1990) Vol. 168, No. 2, 771-777

### SUMMARY OF INVENTION

Therefore, an object of the present invention is to provide a protein having glucocerebrosidase activity even when a sugar chain is not added by post-translational modification.

The present inventors have conducted intensive studies in view of the above problems. As a result, the present inventors have found that the above problems can be solved by the following protein and the like, and have completed the present invention:

A protein, (a) containing an amino acid sequence set forth in SEQ ID NO: 1 or 2 or an amino acid sequence having identity of 90% or more therewith,
(b) being added with no sugar chain, and
(c) having glucocerebrosidase activity.

### DESCRIPTION OF EMBODIMENTS

Hereinafter, embodiments according to the present invention will be described in detail. However, the following descriptions are illustrative for explaining the present invention, and are not intended to limit the technical scope of the present invention to this description range only.

In the present specification, the phrase “X to Y” indicating a range includes X and Y and means “X or more and Y or less”. Unless otherwise specified, operations and measurements of physical properties and the like are measured under the conditions of room temperature (20 to 25° C.)/relative humidity of 40 to 50% RH.

In the present specification, the “glucocerebrosidase activity” means an activity of hydrolyzing glucocerebroside. The presence or absence of glucocerebrosidase activity is determined based on the presence or absence of enzyme reactivity with a synthetic substrate (p-nitrophenyl-β-D-glucopyranoside) described in the section of EXAMPLES described later. The specific activity of the protein after a refolding treatment according to the present invention is, for example, 0.5 U/mg or more, preferably 0.6 U/mg or more, and more preferably 1.2 U/mg or more.

The mature protein of glucocerebrosidase is a polypeptide consisting of 497 amino acid residues generated by cleavage of a propeptide from a precursor protein consisting of 536 amino acid residues. Examples of biopharmaceuticals of glucocerebrosidase put on the market with Gaucher disease as an indication include Cerezyme (registered trademark) (produced from Chinese hamster ovary (CHO) cells), VPRIV (registered trademark) (produced from human fibrosarcoma cells (HT1080)), and Elelyso (registered trademark) (produced from plant (carrot) cells).

One embodiment of the present invention is a protein containing an amino acid sequence set forth in SEQ ID NO: 1 (corresponding to an amino acid sequence of Cerezyme; the amino acid at a position corresponding to position 495 is histidine (H) unlike a human wild-type GBA protein), being added with no sugar chain, and having glucocerebrosidase activity. The amino acid sequence is shown below, and a base sequence (including a termination codon) of a gene (cDNA) encoding the amino acid sequence is shown in SEQ ID NO: 134. In the present specification, a gene encoding the amino acid sequence set forth in SEQ ID NO: 1 is also simply referred to as “GBA gene”.

[Chemical Formula 1]
(SEQ ID NO: 1)
ARPCIPKSFGYSSVVCVCNATYCDSFDPPTFPALGTFSRYESTRS
GRRMELSMGPIQANHTGTGLLLTLQPEQKFQKVKGFGGAMTDAAA
LNILALSPPAQNLLLKSYFSEEGIGYNIIRVPMASCDFSIRTYTY
ADTPDDFQLHNFSLPEEDTKLKIPLIHRALQLAQRPVSLLASPWT
SPTWLKTNGAVNGKGSLKGQPGDIYHQTWARYFVKFLDAYAEHKL
QFWAVTAENEPSAGLLSGYPFQCLGFTPEHQRDFIARDLGPTLAN
STHHNVRLLMLDDQRLLLPHWAKVVLTDPEAAKYVHGIAVHWYLD
FLAPAKATLGETHRLFPNTMLFASEACVGSKFWEQSVRLGSWDRG
MQYSHSIITNLLYHVVGWTDWNLALNPEGGPNWVRNFVDSPIIVD
ITKDTFYKQPMFYHLGHFSKFIPEGSQRVGLVASQKNDLDAVALM
HPDGSAVVVVLNRSSKDVPLTIKDPAVGFLETISPGYSIHTYLWH
RQ

One embodiment of the present invention also includes a protein containing, in place of the amino acid sequence set forth in SEQ ID NO: 1, an amino acid sequence set forth in SEQ ID NO: 2 (corresponding to the amino acid sequence of VPRIV; the amino acid at a position corresponding to position 495 is arginine (R) unlike a human wild-type GBA protein).

[Chemical Formula 2]
(SEQ ID NO: 2)
ARPCIPKSFGYSSVVCVCNATYCDSFDPPTFPALGTFSRYESTRS
GRRMELSMGPIQANHTGTGLLLTLQPEQKFQKVKGFGGAMTDAAA
LNILALSPPAQNLLLKSYFSEEGIGYNIIRVPMASCDFSIRTYTY
ADTPDDFQLHNFSLPEEDTKLKIPLIHRALQLAQRPVSLLASPWT
SPTWLKTNGAVNGKGSLKGQPGDIYHQTWARYFVKFLDAYAEHKL
QFWAVTAENEPSAGLLSGYPFQCLGFTPEHQRDFIARDLGPTLAN
STHHNVRLLMLDDQRLLLPHWAKVVLTDPEAAKYVHGIAVHWYLD
FLAPAKATLGETHRLFPNTMLFASEACVGSKFWEQSVRLGSWDRG
MQYSHSIITNLLYHVVGWTDWNLALNPEGGPNWVRNFVDSPIIVD
ITKDTFYKQPMFYHLGHFSKFIPEGSQRVGLVASQKNDLDAVALM
HPDGSAVVVVLNRSSKDVPLTIKDPAVGFLETISPGYSIHTYLWR
RQ

One embodiment of the present invention further includes a protein containing, in place of the amino acid sequence set forth in SEQ ID NO: 1 or 2, an amino acid sequence having identity (synonymous with “homology” in the present specification) of 90% or more therewith (more preferably, with the amino acid sequence of SEQ ID NO: 1). The identity of SEQ ID NO: 1 with the amino acid sequence set forth in SEQ ID NO: 1 or SEQ ID NO: 2 is more preferably 95% or more and even more preferably 99% or more.

According to the present invention, there is provided a protein having glucocerebrosidase activity even when a sugar chain is not added by post-translational modification.

In the present specification, the identity of the amino acid sequence can be determined using an analysis program such as BLAST, FASTA, or CLUSTAL W. In the case of using BLAST, a default parameter of the program is used.

Here, the “identity” of the amino acid sequence is expressed in percentage as follows: two amino acid sequences to be compared are arranged in parallel such that the amino acid residues of both the amino acid sequences match as many times as possible, and then the number of matched amino acid residues is divided by the total number of amino acid residues. In the alignment, a gap is appropriately inserted into one or both of the two sequences to be compared as necessary, and one inserted gap is counted as one amino acid residue to determine the total number of amino acid residues. When the total number of amino acid residues thus determined is different from between the two sequences to be compared, the sequence identity (%) is calculated by dividing the number of matched amino acid residues by the total number of amino acid residues of the longer sequence.

From the viewpoint that glucocerebrosidase activity can be further improved, a preferred embodiment of the present invention is a protein containing an amino acid sequence having at least one of the following amino acid substitutions in an amino acid sequence of SEQ ID NO: 1 or 2:
(1) a substitution of an amino acid at a position corresponding to position 26 with leucine (F26L);
(2) a substitution of an amino acid at a position corresponding to position 26 with isoleucine (F26I);
(3) a substitution of an amino acid at a position corresponding to position 126 with threonine (C126T);
(4) a substitution of an amino acid at a position corresponding to position 126 with serine (C126S) and a substitution of an amino acid at a position corresponding to position 342 of SEQ ID NO: 1 or 2 with serine (C342S);
(5) a substitution of an amino acid at a position corresponding to position 57 with cysteine (Q57C);
(6) a substitution of an amino acid at a position corresponding to position 60 with cysteine (H60C);
(7) a substitution of an amino acid at a position corresponding to position 63 with cysteine (T63C);
(8) a substitution of an amino acid at a position corresponding to position 143 with cysteine (Q143C);
(9) a substitution of an amino acid at a position corresponding to position 145 with cysteine (H145C);
(10) a substitution of an amino acid at a position corresponding to position 224 with cysteine (K224C); and
(11) a substitution of an amino acid at a position corresponding to position 321 with cysteine (K321C).

The protein according to the present invention is more preferably a protein containing an amino acid sequence having at least one of the following amino acid substitutions in an amino acid sequence of SEQ ID NO: 1 or 2:
(a-1) (1) F26L;
(a-2) (3) C126T;
(a-3) (2) F26I and (3) C126T;
(a-4) (1) F26L and (3) C126T;
(a-5) (4) C126S and C342S;
(a-6) (3) C126T and (5) Q57C;
(a-7) (3) C126T and (6) H60C;
(a-8) (3) C126T and (7) T63C;
(a-9) (3) C126T and (8) Q143C;
(a-10) (3) C126T and (9) H145C;
(a-11) (3) C126T and (10) K224C;
(a-12) (3) C126T and (11) K321C.

Provided that, in (a-1) to (a-12), amino acids at the following positions are not substituted:
in (a-2), an amino acid at a position corresponding to position 142;
in (a-2), an amino acid at a position corresponding to position 144;
in (a-2), an amino acid at a position corresponding to position 147;
in (a-2), an amino acid at a position corresponding to position 171;
in (a-2), an amino acid at a position corresponding to position 347;
in (a-2), an amino acid at a position corresponding to position 407;
in (a-4), an amino acid at a position corresponding to position 248;
in (a-9), an amino acid at a position corresponding to position 77;
in (a-9), an amino acid at a position corresponding to position 290;
in (a-9), an amino acid at a position corresponding to position 293;
in (a-9), an amino acid at a position corresponding to position 333; and
in (a-9), an amino acid at a position corresponding to position 466.

Examples of the amino acid sequence having at least one of the above-described amino acid substitutions include amino acid sequences set forth in SEQ ID NOs: 3 to 5, 7, 9 to 30, 32, 33, 35, 37 to 39, and 41 to 51.

Still more preferably, the protein according to the present invention contains at least one selected from amino acid sequences set forth in SEQ ID NOS: 3, 5, 7, 9, 10, 12 to 16, 18 to 28, 30, 32, 33, 35, 37 to 39, 42, and 47.

From the viewpoint that stability can be further improved, a preferred embodiment of the present invention is desirably a protein containing at least one of the following amino acid substitutions in an amino acid sequence of SEQ ID NO: 1 or 2:
(12) a substitution of an amino acid at a position corresponding to position 248 with serine (C248S) and a substitution of an amino acid at a position corresponding to position 342 with serine (C342S);
(13) a substitution of an amino acid at a position corresponding to position 126 with threonine (C126T) and a substitution of an amino acid at a position corresponding to position 342 with serine (C342S);
(14) a substitution of an amino acid at a position corresponding to position 126 with serine (C126S), a substitution of an amino acid at a position corresponding to position 248 with serine (C248S), and a substitution of an amino acid at a position corresponding to position 342 with serine (C342S); and
(15) a substitution of an amino acid at a position corresponding to position 126 with threonine (C126T), a substitution of an amino acid at a position corresponding to position 248 with serine (C248S), and a substitution of an amino acid at a position corresponding to position 342 with serine (C342S).

More desirably, the protein according to the present invention contains at least one selected from amino acid sequence set forth in SEQ ID NOS: 14, 17, 18, and 51.

A preferred embodiment of the present invention is a protein further containing at least one of the following amino acid substitutions in an amino acid sequence of SEQ ID NO: 1 or 2 or an amino acid sequence of SEQ ID NO: 1 or 2 having any one of the substitutions (1) to (15) described above:
(16) a substitution of an amino acid at a position corresponding to position 61 with cysteine (T61C);
(17) a substitution of an amino acid at a position corresponding to position 98 with cysteine (P98C);
(18) a substitution of an amino acid at a position corresponding to position 143 with cysteine (Q143C);
(19) a substitution of an amino acid at a position corresponding to position 224 with cysteine (K224C);
(20) a substitution of an amino acid at a position corresponding to position 321 with cysteine (K321C); and
(21) a substitution of an amino acid at a position corresponding to position 407 with cysteine (T407C).

More preferably, the protein according to the present invention contains at least one selected from amino acid sequences set forth in SEQ ID NOs: 16, 24, 28, 30, 37, 39, 41, and 43 to 49.

The protein according to the present invention may be a protein consisting of the amino acid sequence described above.

An example of a method for producing a protein of the present invention will be described below.

A method for producing a peptide chain as a protein raw material according to the present invention is not particularly limited as long as a sugar chain is not added to a peptide chain, and the peptide chain may be a peptide chain produced by a prokaryote or a peptide chain synthesized by organic synthesis. The protein according to the present invention, from the viewpoint of high productivity and low cost, preferably, a peptide chain produced by a prokaryote can be used as a raw material.

That is, in one embodiment, the protein according to the present invention is produced by a prokaryote.

Examples of the prokaryote include bacteria belonging to the genus Escherichia such as Escherichia coli, the genus Bacillus such as Bacillus subtilis, the genus Pseudomonas such as Pseudomonas putida, and the genus Rhizobium such as Rhizobium meliloti. The prokaryote used in the present invention is preferably E. coli.

In one embodiment, the method for producing a protein according to the present invention includes introducing a vector containing a nucleic acid encoding the protein according to the present invention into a prokaryote to cause the prokaryote to produce a protein raw material, and subjecting the protein raw material which is collected to a folding treatment.

First, the vector containing a nucleic acid encoding the protein according to the present invention is introduced into a prokaryote, and the prokaryote is caused to produce a protein raw material. Thereby, a protein raw material added with no sugar chain can be obtained.

Methods for producing a nucleic acid encoding the protein according to the present invention and a vector containing the nucleic acid are not particularly limited, and conventionally known methods can be used.

As the vector, a known vector, for example, a T vector such as pTAKN-2, or a plasmid vector such as pET-21b(+) can be used.

A method for introducing a vector into a prokaryote is not particularly limited, and a conventionally known method can be appropriately used. Examples of the introduction method include a competent cell method, a conjugate transfer method, a calcium phosphate method, a lipofection method, an electroporation method, and the like.

By culturing the prokaryote into which the vector has been introduced, the prokaryote can be caused to produce a protein raw material. Culturing the prokaryote can be carried out according to the usual method used for a selected prokaryote.

Depending on the type of prokaryotes to be used, a prokaryote is cultured under aerobic or anaerobic conditions. In the former case, the prokaryote may be cultured by shaking, aeration stirring, or the like. The culture conditions (culture temperature, culture time, pH of medium, and the like) are appropriately selected depending on the composition of a medium and a culture method, and are not particularly limited as long as the prokaryote can grow, and can be appropriately selected according to the type of prokaryote to be cultured.

Since the protein according to the present invention is not added with a sugar chain by post-translational modification, that is, it is desired that the protein is not subjected to post-translational modification.

A method for collecting a protein raw material produced by a prokaryote is not particularly limited, and a conventionally known method can be appropriately used. For example, when the protein raw material is present in a prokaryote, the prokaryote is collected from the obtained culture by a method such as centrifugation or filtration, and the collected prokaryote is disrupted by a mechanical method using beads or the like or an enzymatic method. After crushing, the insoluble fraction is collected and treated with a buffer containing a surfactant, whereby the protein raw material can be collected.

Next, the collected protein raw material is subjected to a folding treatment (may be a refolding treatment including a denaturation treatment performed in advance).

The folding treatment can be performed, for example, by adding a buffer containing an oxidizing agent and a reducing agent (oxidized glutathione/reduced glutathione, cystine/cysteine, cysteamine/cystamine, or the like) to a liquid containing the collected protein raw material and allowing the mixture to stand still at about 20° C. to about 30° C. for about 1 day to 7 days. An additive such as sucrose or glycerol can be further added to the buffer.

The collected protein raw material may be subjected to a denaturation (solubilization) treatment as a necessary before the folding treatment. The denaturation treatment can be performed using a denaturant such as 6 M guanidine hydrochloride or 8 M urea. By performing the denaturation treatment, the collected protein raw material can be brought into an unfolded state.

In one embodiment, the method for producing a protein according to the present invention is a method for producing a protein having glucocerebrosidase activity, the method including subjecting a protein raw material, which contains an amino acid sequence constituting the unfolded protein according to the present invention and is added with no sugar chain, to a folding treatment.

In one embodiment, the protein according to the present invention is produced by refolding a protein produced by a prokaryote. The protein produced by the prokaryote may be subjected to a denaturation (solubilization) treatment as necessary.

Unlike a conventional recombinant GBA protein using an animal cell and a plant cell, the protein according to the present invention can suppress the risk of viral infection and is also expected to suppress antigenicity to a mammal.

The protein according to the present invention is also suitable for the following use applications.

Even when a recombinant GBA protein produced by a prokaryote is used as a raw material, a recombinant GBA protein having activity can be provided. Therefore, the protein according to the present invention can be suitably used in the treatment of lysosomal diseases such as Gaucher disease.

The protein according to the present invention can be used for degrading glucosylceramides such as those derived from plants and producing ceramides.

The protein according to the present invention can be used for obtaining a GBA antibody.

### EXAMPLES

Hereinafter, the present invention will be specifically described with reference to Examples, but the present invention is not limited thereto. In particular, it is possible to appropriately use other generally known means for portions (1-1 to 2-2) related to the construction and culture of a bacterial strain producing a GBA protein and a recombinant GBA protein and the disruption of cells.

In Examples, the plasmid number and the recombinant protein number are given the same number.

[Construction of GBA Gene and Altered Gene-Introduced Recombinant E. coli Thereof]

### 1-1. Synthesis of Glucocerebrosidase (GBA) Gene

A GBA gene represented by SEQ ID NO: 135 is obtained by adding an initiation codon (atg) to the 5′-end of a codon encoding a mature GBA protein from which a signal peptide has been removed, and by making a change so as to obtain a sequence optimized for codon usage frequency of E. coli (E. coli K-12 strain), The synthesis of the GBA gene represented by SEQ ID NO: 135 was outsourced to Eurofins Genomics K. K., and delivered in a state of being inserted into pTAKN-2 containing an ampicillin resistance gene.

1-2. Preparation of Plasmid into which GBA Gene is Inserted

For expression studies in E. coli, the GBA gene obtained above was subcloned between the NdeI site and the His tag of the pET-21b(+) plasmid vector (Novagen). Specifically, PCR using either pET-21b(+) or pTAKN-2 into which the GBA gene was inserted as a template was performed to obtain an amplification product of linearized pET-21b(+) and the GBA gene (excluding a termination codon).

TABLE 1
Sequence of primer
For pET-21b(+) amplification

| | |
|---|---|
| Forward primer | 5′-CAGTAAGGATCCGA |
| (SEQ ID NO: 130) | ATTCGAGCTCCG-3′ |
| Reverse primer | 5′-TGATGATGATGGCC |
| (SEQ ID NO: 131) | CATGGTATATCTCCTTC |
| | TTAAAGTTAAACAAAAT |
| | TATTTCTAGAG-3′ |

For pTAKN-2 amplification

| | |
|---|---|
| Forward primer | 5′-GGGCCATCATCATC |
| (SEQ ID NO: 132) | ATCATCATCATCATCAT |
| | CACAGCAGCGGCCATAT |
| | CGACGACGACGACAAGG |
| | CGCGCCCATGTATCC |
| | C-3′ |
| Reverse primer | 5′-TTCGGATCCTTACT |
| (SEQ ID NO: 133) | GACGATGCGACAGATAG |
| | GTG-3′ |

The PCR amplification product obtained above was subjected to a treatment (cleavage by the restriction enzyme DpnI and ligation) using In-Fusion HD Cloning Kit (Takara Bio Inc.) to obtain a pET-21b(+) plasmid vector into which the GBA gene was inserted (referred to as “H495 type” in the present specification). The GBA gene inserted into the plasmid vector encodes the amino acid sequence set forth in SEQ ID NO: 1.

1-3. Preparation of Plasmid into which Altered GBA Gene is Inserted

PCR using a plasmid into which the GBA gene prepared in the above 1-2. was inserted as a template was performed using a primer for mutation introduction (intended to substitute the amino acid encoded by the GBA gene with another amino acid) described in Table 1 below to variously amplify a plasmid (linearized plasmid) in which a mutation was introduced into the GBA gene (excluding the termination codon). Substitution sites of the amino acid sequence and codons corresponding to the substituted amino acids in the various altered GBA genes are as shown in Table 2. According to the manual, the obtained PCR amplification product (linearized plasmid) was self-ligated and circularized with T4 Polynucleotide Kinase (TOYOBO Co., Ltd.) and Ligation high Ver. 2 (TOYOBO Co., Ltd.) to obtain a plasmid into which the altered GBA gene was inserted (Table 5). When a plurality of mutations were introduced, mutations were added by repeating the same method as described above.

TABLE 1

| SEQ ID | Altered | | |
|---|---|---|---|
| NO: | site | | |
| 52 | F26L | Forward primer | 5′-ctgGATCCGCCCACTTTTCCC-3′ |
| 53 | | Reverse primer | 5′-CGAATCGCAGTACGTGGC-3′ |
| 54 | C126S | Forward primer | 5′-agcGACTTTTCGATTCGCACCTATAC-3′ |
| 55 | | Reverse primer | 5′-CGAGGCCATTGGAACGCG-3′ |
| 56 | C126T | Forward primer | 5′-CGAGGCCATTGGAACGCG-3′ |
| 57 | | Reverse primer | 5′-ggtCGAGGCCATTGGAACGCG-3′ |
| 58 | C342G | Forward primer | 5′-GTGGGGAGCAAGTTTTGGGAG-3′ |
| 59 | | Reverse primer | 5′-gccTGGTTCGCTAGCAAAGAGCATC-3′ |
| 60 | C342S | Forward primer | 5′-AAGGAagcGTGGGGAGCAAGTTTTGG-3′ |
| 61 | | Reverse primer | 5′-CCCACgctTGCTTCGGTAGCAAAGAGC-3′ |
| 62 | C342T | Forward primer | 5′-AAGCAcccGTGGGGAGCAAGTTTTGG-3′ |
| 63 | | Reverse primer | 5′-CCCACggtTGCTTCGCTAGCAAAGAGC-3′ |
| 64 | F26I | Forward primer | 5′-attGATCCGCCCACTTTTCCC-3′ |
| 65 | | Reverse primer | 5′-GGAATCGAGTACGTGGC-3′ |
| 66 | C248S | Forward primer | 5′-agcCTGGGTTTTACGCCGGAAC-3′ |
| 67 | | Reverse primer | 5′-TTGAAACGGATAACCCGAGAG-3′ |
| 68 | S184C | Forward primer | 5′-tgcTTACCGGAAGAGGATACGAAAC-3′ |
| 69 | | Reverse primer | 5′-GAAATTGTGCAGCTGAAAGTCATC-3′ |
| 70 | T61C | Forward primer | 5′-tgcGGTACTGGGCTGTTGCTTAC-3′ |
| 71 | | Reverse primer | 5′-GTGATTGGCCTGAATAGGGC-3′ |
| 72 | I56C | Forward primer | 5′-CAGGCCAATCACACGGTAC-3′ |
| 73 | | Reverse primer | 5′-gcaAGGGCCGATAGACAGTTCC-3′ |
| 74 | Q857C | Forward primer | 5′-GCCAATCACACCGGTACTG-3′ |
| 75 | | Reverse primer | 5′-gcaAATAGGGCCCATAGACAGTTC-3′ |
| 76 | A58C | Forward primer | 5′-AATCACACCGGTACTGGG-3′ |
| 77 | | Reverse primer | 5′-gcaCTGAATAGGGCCCATAGAC-3′ |
| 78 | N59C | Forward primer | 5′-AGGGCTGCtgcCACACCGGTACTGGGCTG-3′ |
| 79 | | Reverse primer | 5′-GAGAAgcaGTGCAGCTGAAAGTCATC-3′ |
| 80 | H6DC | Forward primer | 5′-ACCGGTACTGGGCTGTTG-3′ |
| 81 | | Reverse primer | 5′-gcaATTGGCCTGAATAGGGCG-3′ |
| 82 | T61C | Forward primer | 5′-tgcGGTACTGGGCTGTTGCTTAC-3′ |
| 83 | | Reverse primer | 5′-GTGATTGGCCTGAATAGGGC-3′ |
| 84 | Q62C | Forward primer | 5′-ACTGGGCTGTTGCTTACC-3′ |
| 85 | | Reverse primer | 5′-gcaGGTGTGATTGGCCTGAATAG-3′ |
| 86 | T63C | Forward primer | 5′-GGGCTGTTGCTTACCCTC-3′ |
| 87 | | Reverse primer | 5′-gcaACGGGTGTGATTGGCCTG-3′ |
| 88 | G64C | Forward primer | 5′-CTGTTGCTTACCCTCCAAC-3′ |
| 89 | | Reverse primer | 5′-gcaAGTACCGGTGTGATTGGC-3′ |
| 90 | P98C | Forward primer | 5′-tgcCTGGGTTTTACGCCGGAAC-3′ |
| 91 | | Reverse primer | 5′-TTGAAACGGATAACCCGAGAG-3′ |
| 92 | F142C | Forward primer | 5′-CAGCTGCACAATTTCTCGTTAC-3′ |
| 93 | | Reverse primer | 5′-gcaGTCATCCGGCGTATCCGC-3′ |
| 94 | Q143C | Forward primer | 5′-CTGCACAATTTCTCGTTACC-3′ |
| 95 | | Reverse primer | 5′-gcaAAAGTCATCCGGCGTATC-3′ |
| 96 | L144C | Forward primer | 5′-CACAATTTCTCTCGTTAGCGGAAG-3′ |
| 97 | | Reverse primer | 5′-gcaCTGAAAGTCATCCGGCGTATC-3′ |
| 98 | H145C | Forward primer | 5′-AATTTCTGTGGTTAGCGGAAGAGG-3′ |
| 99 | | Reverse primer | 5′-gcaCAGCTGAAAGTCATCCGGC-3′ |
| 100 | N146O | Forward primer | 5′-TGCACtgcTTCTCGTTACCGGAAGAG-3′ |
| 101 | | Reverse primer | 5′-GTGTGgcaGGCCTGAATAGGGCCCATAG-3′ |
| 102 | F147C | Forward primer | 5′-TCGTTACCGGAAGAGGATACG-3′ |
| 103 | | Reverse primer | 5′-gcaATTGTGCAGCTGAAAGTCATC-3′ |
| 104 | S148C | Forward primer | 5′-tgcTTACCGGAAGAGGATACGAAAC-3′ |
| 105 | | Reverse primer | 5′-GAAATTGTGCAGCTGAAAGTCATC-3′ |
| 106 | P171C | Forward primer | 5′-GTGTCCCTGCTGGCTTCAC-3′ |
| 107 | | Reverse primer | 5′-gcaACGCTGTCGGAATTGTAAC-3′ |
| 108 | K224C | Forward primer | 5′-CTGCAGTTTTGGGCCGTG-3′ |
| 109 | | Reverse primer | 5′-gcaATGCTCAGCATACGCATC-3′ |
| 110 | P299C | Forward primer | 5′-GAAGCTGCGAAATATGTG-3′ |
| 111 | | Reverse primer | 5′-gcaATCGGTTAACACCACTTTG-3′ |
| 112 | K321C | Forward primer | 5′-GCCACCTTGGGAGAAACTC-3′ |
| 113 | | Reverse primes | 5′-gcaGGGAGGCGCCAGAAAATC-3′ |
| 114 | F347C | Forward primer | 5′-cTGGGAGCAAAGCGTCCG-3′ |
| 115 | | Reverse primer | 5′-caCTTGCTGGCGACgctTG-3′ |
| 116 | T407C | Forward primer | 5′-AAGGATACGTTCTACAAACAGC-3′ |
| 117 | | Reverse primer | 5′-gcaGATATCGACAATGATCGGAGAATC-3′ |
| 118 | K441C | Forward primer | 5′-AACGACCTAGATGCAGTAGC-3′ |
| 119 | | Reverse primer | 5′-gcaCTGGGAGGCTACCAGGCC-3′ |
| 120 | K77C | Forward primer | 5′-GTGAAAGGCTTTGGCGGC-3′ |
| 121 | | Reverse primer | 5′-gcaCTGAAATTTCTGCTCCGGTTGG-3′ |
| 122 | H290C | Forward primer | 5′-TGGGCCAAAGTGGTGTTAAC-3′ |
| 123 | | Reverse primer | 5′-gcaCGGAAGGAGCAACCGTTG-3′ |
| 124 | T297C | Forward primer | 5′-GATCCAGAAGCTGCGAAATATG-3′ |
| 125 | | Reverse primer | 5′-gcaTAACACCACTTTGGCCCAAT-3′ |
| 126 | N333C | Forward primer | 5′-ACGATGCTCTTTGCTAGCGA-3′ |
| 127 | | Reverse primer | 5′-gcaCGGGAAAGAGGCGATGAGTTTC-3′ |
| 128 | K466C | Forward primer | 5′-GACGTACCGCTGACCATC-3′ |
| 129 | | Reverse primer | 5′-gcaTGAACTGCGGTTTAAAACAAC-3′ |

TABLE 5
(Table 2)

| Amino acid residue after | |
|---|---|
| substitution | Codon |
| R | cgc |
| S | agc |
| T | acc |
| C | tgc |
| G | ggc |
| I | att |
| L | ctg |

1-4. Construction of Recombinant E. coli Strain

Each of the plasmids constructed in 1-2 and 1-3 was transformed into a competent cell of E. coli (ECOS competent 10 E. coli BL21 (DE3) (NIPPON GENE CO., LTD.)) according to the manual, and various recombinant E. coli strains retaining a plasmid vector into which a GBA gene or an altered GBA gene was inserted were constructed.

[Synthesis Method of Protein by Recombinant E. coli, Comparative Evaluation Method, and Comparative Evaluation Result]
2-1. Synthesis of GBA Protein by Recombinant E. coli

A GBA protein or a recombinant GBA protein was synthesized using the recombinant E. coli constructed in the above 1-4.

Specifically, first, a single colony grown on an LB agar medium (containing ampicillin at a concentration of 100 mg/L) was inoculated into 4 mL of an LB liquid medium (containing ampicillin at a concentration of 100 mg/L) in a test tube, and shaken and cultured at 300 rpm and 30° C. overnight to obtain a preculture solution.

The preculture solution (2 mL) was inoculated into 50 mL of the medium for main culture (see Table 3 below for the composition) in a Sakaguchi flask, and shaken and cultured at 120 rpm and 30° C. for 72 hours to perform main culture.

TABLE 6
(Table 3) Medium for main culture

| | | |
|---|---|---|
| Glycerol | 40 | g/L |
| (NH₄)₂SO₄ | 10 | g/L |
| KH₂PO₄ | 2 | g/L |
| K₂HPO₄ | 6 | g/L |
| Yeast extract | 40 | g/L |
| MgSO₄•7H₂O | 1 | g |
| ADEKANOL | 0.5 | mL/L |
| Ampicillin | 100 | mg/L |
| IPTG | 100 | μM |

The culture solution after the main culture was centrifuged at 6,000*g and 4° C. for 10 minutes, the supernatant was discarded, and then a precipitate was suspended using buffer A (see Table 4 below for the composition). Thereafter, the resultant solution was centrifuged again at 6,000*g and 4° C. for 10 minutes, and the supernatant was discarded to obtain a precipitate of recombinant E. coli (followed by cryopreservation at −80° C.).

TABLE 7
(Table 4) Buffer A

| | | |
|---|---|---|
| D-mannitol | 32 | g/L |
| Trisodium citrate dihydrate | 16.54 | g/L |
| Citrate monohydrate | 1.28 | g/L |
| Tween 80 | 140 | mg/L |

### 2-2. Crushing Treatment of Bacterial Cells

The recombinant E. coli obtained in the above 2-1 was suspended in buffer A, the turbidity (OD660) was measured, and then dilution with buffer A was performed so that OD660 was 10. Zirconia silica beads (0.6 mm) were added to this suspension, and the mixture was shaken at 1300 rpm for 5 minutes by a bead-based cell disruptor (Shake Master Neo ver 1.0 manufactured by Bio Medical Science Inc.) while being cooled using an aluminum block cooled on ice, and then further cooled with an aluminum block for 5 minutes. This operation was repeated six times in total, and the cells of the bacterial cells were subjected to a crushing treatment.

Next, the resultant solution was centrifuged at 6,000×g and 4° C. for 15 minutes, and a precipitate (insoluble fraction) was collected. Each of the following solutions (1) to (4) (200 μL) was sequentially subjected to suspension and then a centrifugation treatment at 6000×g for 2 minutes twice for the collected insoluble fraction to obtain an insoluble protein.
(1) Buffer A
(2) 0.05 w/v % sodium deoxycholate (DOC·Na)-added buffer A
(3) 1 w/v % Triton X-100-added buffer A
(4) Buffer A (pH 6)

### 2-3. Denaturation (Solubilization) Treatment

Subsequently, the insoluble protein obtained by the centrifugation treatment was suspended in a 20 mM potassium phosphate buffer (pH 8) to which 6 M guanidine hydrochloride, 0.014 w/v % Tween 80, and 40 mM dithiothreitol (DTT) were added, and then allowed to stand still at 25° C. for 2 hours for incubation (denaturation (solubilization) treatment).

Next, the mixture was centrifuged at 6,000×g and 4° C. for 10 minutes, and the insoluble component was removed by collecting the supernatant. The absorbance (280 nm) of the solution was measured using a spectrophotometer, and the protein was quantified from the obtained value (A280) according to the mathematical formula of protein concentration (mg/mL)=A280/1.7. The denominator 1.7 is an absorption coefficient calculated based on amino acid sequence information.

### 2-4. Refolding Treatment

Thereafter, a solution was prepared using a 20 mM potassium phosphate buffer (pH 8) to which 6 M guanidine hydrochloride and 0.014 w/v % Tween 80 were added so as to have a protein concentration of 1 mg/mL, and then diluted 50 times with an added 20 mM potassium phosphate buffer (pH 8) to which 40 w/v % glycerol, 0.25 w/v % Tween 80, 3 mM oxidized glutathione (GSSG), and 6 mM reduced glutathione (GSH) were added.

Incubation was started by allowing the resultant solution to stand still at 25° C. from the time point of dilution, the sample was collected 7 days after the start of incubation, and the enzyme activity was measured by the following method.

### 2-5. Measurement of Enzyme Activity

The glucocerebrosidase (GBA) is an enzyme that catalyzes a reaction of hydrolyzing dehydration-condensation sites between sugar and lipid of Glc-Cer (glucocerebroside; glycolipid). Herein, the enzyme activity of the recombinant GBA protein obtained above was measured using p-nitrophenyl-β-D-glucopyranoside (pNPG), which is a synthetic substrate, as a substrate.

Specifically, first, 90 μL of 1 w/v % Triton X-100-added buffer A, 30 μL of the sample (after 7 days), and 30 μL of 50 mM pNPG-added buffer A were mixed, and the mixture was incubated at 700 rpm and 37° C. for 1 hour using a thermomixer comfort (Eppendorf). Then, 150 μL of a 0.2 N NaOH solution was added, vortexed, and then centrifuged at about several thousand rpm at room temperature for several seconds.

The supernatant (200 μL) was transferred to a microplate and the absorbance (400 nm) corresponding to the reaction product (4-nitrophenol) was measured. The capacity activity (U/mL) of the recombinant GBA protein was calculated based on a calibration curve of 4-nitrophenol prepared in advance. The specific activity (U/mg) of the recombinant GBA protein was calculated by dividing the value of the capacity activity by the set protein concentration (20 mg/L). Note that “1 U” is a unit of activity that degrades pNPG by 1 μmol per minute. For a GBA protein containing an amino acid sequence of SEQ ID NO: 1 (referred to as “H495 type protein” in the present specification) produced by E. coli in the same manner as described above using the plasmid into which the GBA gene prepared in the above 1-2. was inserted, the refolding treatment and the measurement of enzyme activity were also performed in the same manner as described above. The specific activity of the H495 type protein was 1.2 U/mg.

The results of enzyme activity measurement are shown in Table 5 below. Here, the values shown in Table 5 are relative values (%) when the specific activity value of the H495 type protein is taken as 100%.

The measurement of the enzyme activity in the present specification is performed according to the above-described method unless otherwise specified.

TABLE 8
(Table 5)
[Table 9]

| No. | Altered site | Relative activity (%)* | |
|---|---|---|---|
| H495 type | | 100 | Example |
| No. 142 | [symbol] | [symbol] | Example |
| [symbol] | [symbol] | 78 | Example |
| No. 145 | [symbol] | 248 | Example |
| No. 38 | [symbol] | 1 | Comparative Example |
| No. 3 | [symbol] | 101 | Example |
| No. 4 | [symbol] | 1 | Comparative Example |
| [symbol] | [symbol] | [symbol] | Example |
| No. 159 | [symbol] | 258 | Example |
| No. 184 | [symbol] | 50 | Example |
| No. 185 | [symbol] | [symbol] | Example |
| [symbol] | [symbol] | [symbol] | Example |
| [symbol] | [symbol] | [symbol] | Example |
| No. 147 | [symbol] | 215 | Example |
| No. 149 | [symbol] | 274 | Example |
| [symbol] | [symbol] | 70 | Example |
| No. 167 | [symbol] | [symbol] | Example |
| [symbol] | [symbol] | [symbol] | Example |
| No. 187 | [symbol] | [symbol] | Example |
| No. 188 | [symbol] | [symbol] | Example |
| No. 189 | [symbol] | 121 | Example |
| No. 190 | [symbol] | 148 | Example |
| [symbol] | [symbol] | 130 | Example |
| No. 191 | [symbol] | [symbol] | Example |
| No. 192 | [symbol] | [symbol] | Example |
| No. 193 | [symbol] | 123 | Example |
| [symbol] | [symbol] | [symbol] | Example |
| No. 171 | [symbol] | [symbol] | Example |
| [symbol] | [symbol] | [symbol] | Example |
| No. 172 | [symbol] | 12 | Comparative Example |
| No. 173 | [symbol] | 142 | Example |
| [symbol] | [symbol] | 124 | Example |
| No. 174 | [symbol] | 8 | Comparative Example |
| No. 175 | [symbol] | 123 | Example |
| [symbol] | [symbol] | 34 | Comparative Example |
| No. 194 | [symbol] | 184 | Example |
| No. 195 | [symbol] | 135 | Example |
| No. 198 | [symbol] | [symbol] | Example |
| No. 201 | [symbol] | [symbol] | Comparative Example |
| No. 215 | [symbol] | 85 | Example |
| No. 200 | [symbol] | 141 | Example |
| No. 178 | [symbol] | 92 | Example |
| No. 252 | [symbol] | [symbol] | Example |
| No. 254 | [symbol] | 63 | Example |
| No. 257 | [symbol] | 75 | Example |
| No. 259 | [symbol] | 103 | Example |
| No. 263 | [symbol] | 72 | Example |
| No. 243 | [symbol] | 84 | Example |

[symbol]

indicates data missing or illegible when filed

### DISCUSSION

It has been newly found that the H495 type protein has an enzyme activity even though the sugar chain modification was not performed.

From the comparison between the H495 type protein and No. 142, the comparison between No. 145 and No. 159, and the comparison between No. 147 and No. 149, the activity improving effect of F26L was found.

From the comparison between No. 145 and No. 165, the activity improving effect of F26I was found.

From the comparison between No. 18 and No. 145, the comparison between No. 27 and No. 125, the comparison between No. 184 and No. 185, and the comparison between No. 37 and No. 167, the activity improving effect of C126T was found.

From the comparison of No. 3, No. 18, and No. 27, the activity improving effect of C342S and C126S was found.

From the comparison of No. 167, No. 168, and Nos. 186 to 193, the activity improving effect of Q57C, H60C, and T63C was found.

From the comparison between No. 167 and Nos. 171 to 176, the activity improving effect of Q143C and H145C was found.

From the comparison of No. 167, Nos. 194 to 198, No. 200, No. 201, and No. 215, and the comparison of No. 178, No. 243, No. 252, No. 254, No. 257, No. 259, and No. 263, the activity improving effect of K224C and K321C was found.

It has been reported that C342 is an amino acid residue necessary for enzyme activity (THE JOURNAL OF BIOLOGICAL CHEMISTRY VOL. 281, NO. 7, pp. 4242-4253 Feb. 17, 2006). However, it was found that in the case of substitution with serine, the activity was maintained.

### [Stability Evaluation]

Synthesis of glucocerebrosidase variant added with no sugar chain

A plasmid into which a recombinant GBA gene containing the mutations of C248S or C248S and C342S was inserted was additionally obtained by the same method as in the above 1-3 (Nos. 19 and 42). Thereafter, a recombinant E. coli strain retaining the plasmid was also additionally prepared by the same method as in the above 1-4.

The H495 type protein and each recombinant GBA protein were obtained from each recombinant E. coli strain retaining the plasmid described in Table 6 by the same method as in the above 2-1 to 2-3.

### 3-1. Stability Evaluation in Refolding Solution

For the H495 type protein obtained above and seven recombinant GBA proteins in Table 6 below, the solution (sample) after 7 days had passed after 2-4. Refolding treatment described above was transferred to 37° C., and the transition of the residual activity was measured. The results are shown in Table 6.

TABLE 6

| | | Time (h) | | | | |
|---|---|---|---|---|---|---|
| No. | Altered site | 0 | 24 | 48 | 72 | 96 |
| H495 type | — | 100 | 83 | 53 | 19 | 6 |
| No. 18 | C126S | 100 | 90 | 70 | 35 | 12 |
| No. 19 | C248S | 100 | 89 | 69 | 33 | 12 |
| No. 42 | C248S, C342S | 100 | 93 | 86 | 67 | 48 |
| No. 27 | C126S, C342S | 100 | 88 | 70 | 20 | 5 |
| No. 125 | C126T, C342S | 100 | 96 | 94 | 76 | 53 |
| No. 37 | C126S, C248S, C342S | 100 | 87 | 81 | 74 | 67 |
| No. 167 | C126T, C248S, C342S | 100 | 96 | 98 | 97 | 95 |

As shown in Table 6, it was confirmed that stability is improved by substituting a Cys residue, and stability is further improved by substituting a plurality of Cys residues, as compared with the H495 type protein.

### 3-2. Stability Evaluation 1 in Buffer

For the recombinant GBA protein of Table 9 below, the pH was adjusted to 4.5 by adding a 1 M citric acid solution to the solution (sample) after 7 days had passed after 2-4. Refolding treatment described above. Next, the mixture was filtered through a filter sterilizing filter (manufactured by Nalgen, 0.2 μm, PES), and then desalted and concentrated (about 10 times each) by Pellicon 2, Biomax, 10 kDa, 0.1 m², V-screen (Merck). The obtained concentrated solution was purified by HiTrap SP HP, 5 mL (GE Healthcare). A solution A: buffer B (see Table 7 below for the composition) and a solution B: 1 M NaCl-added buffer A were used as solutions, and an active fraction eluted at B 25% was collected. Purification was performed by HiTrap Phenyl HP, 5 mL (GE Healthcare). A solution A: buffer C (see Table 8 below for the composition) and a solution B: ethanol were used as solutions, and an active fraction eluted at B 40% was collected. The collected solution was concentrated with Amicon Ultra-15, 3 kDa (Merck), and then freeze-dried.

TABLE 11
(Table 7) Buffer B

| | | |
|---|---|---|
| D-mannitol | 32 | g/L |
| Trisodium citrate dihydrate | 16.54 | g/L |
| Citrate monohydrate | 9.95 | g/L |
| Tween 80 | 140 | mg/L |

TABLE 12
(Table 8) Buffer C

| | | |
|---|---|---|
| D-mannitol | 6.4 | g/L |
| Trisodium citrate dihydrate | 3.3 | g/L |
| Citrate monohydrate | 0.26 | g/L |
| Tween 80 | 28 | mg/L |

Cerezyme (registered trademark) and purified recombinant GBA protein (No. 176) were diluted with 0.015 w/v % Tween 80-added 20 mM potassium phosphate buffer (pH 7) to 0.05 mg/mL, and incubated at 37° C., and the transition of the residual activity was measured. The results are shown in Table 9.

TABLE 9

| | | Time (h) | | | | | | |
|---|---|---|---|---|---|---|---|---|
| Altered site | | 0 | 1 | 3 | 4 | 5.5 | 21 | 46.2 |
| Cerezyme | — | 100 | 68 | 53 | 47 | 40 | 6 | 0 |
| No. 176 | C126T, C248S, | 100 | 71 | 67 | 65 | 61 | 40 | 23 |
| | C342S, Q143C | | | | | | | |

As shown in Table 9, it was confirmed that the recombinant GBA protein (No. 176) has improved stability with respect to Cerezyme.

### 3-3. Stability Evaluation 2 in Buffer

The recombinant GBA proteins (No. 167 and No. 178) were purified by the same method as in Stability evaluation 1 in the buffer.

Cerezyme (registered trademark) and purified recombinant GBA proteins (No. 167 and No. 178) were diluted with 0.1 w/v % Tween 80-added 50 mM potassium phosphate buffer (pH 7) to 0.01 mg/mL, and incubated at 37° C., and the transition of the residual activity was measured. The results are shown in Table 10.

TABLE 10

| | | Time (h) | | | | | |
|---|---|---|---|---|---|---|---|
| Altered site | | 0 | 2 | 4 | 6 | 24 | 48 |
| Cerezyme | — | 100 | 36 | 16 | 7 | 0 | 0 |
| No.167 | C126T, C248S, C342S | 100 | 47 | 40 | 37 | 12 | 4 |
| No.178 | C126T, C248S, | 100 | 47 | 41 | 36 | 16 | 8 |
| | C342S, T61C, Q143C | | | | | | |

As shown in Table 10, it was confirmed that the recombinant GBA proteins (No. 167 and No. 178) have improved stability with respect to Cerezyme.

### [Study of Refolding Conditions for Recombinant GBA Protein]

### 4-1. Study of Additive (Oxidized Glutathione, Reduced Glutathione)

For the GBA protein (H495 type protein) containing an amino acid sequence of SEQ ID NO: 1 produced by E. coli in the same manner as described above using the wild-type plasmid prepared in the above 1-2., an insoluble protein was prepared by the procedure described in the above “2-2. Crushing treatment of bacterial cells”.

Next, a denatured protein solution of the H495 type protein was prepared by the procedure described in “2-3. Denaturation (solubilization) treatment” for the obtained insoluble protein of the H495 type protein. This solution was diluted with a 20 mM potassium phosphate buffer (pH 8) to which 6 M guanidine hydrochloride and 0.014 w/v& Tween 80 were added, based on the protein concentration calculated from the absorbance (280 nm) of the solution, and the protein concentration was set to about 1 mg/mL. Thereafter, the solution was diluted 50 times with a refolding solution (at the start of refolding: 20 mg/L protein). The composition of the refolding solution was obtained by adding 1 M sucrose, 0 to 3 mM reduced glutathione (GSSG), 0 to 30 mM oxidized glutathione (GSH), and 0.014 w/v % Tween 80 to a 20 mM potassium phosphate buffer (pH 8). At this time, the concentration of GSSG was changed in a range of 0 to 3 mM, and the concentration of GSH was changed in a range of 0 to 30 mM. The results are shown in Table 11. From these results, it was confirmed that the highest enzyme activity was achieved when 3 mM GSSG and 6 mM GSH were used in combination as additives.

TABLE 15
(Table 11)

| | Additive concentration | | Capacity |
|---|---|---|---|
| | GSSG | GSH | activity |
| | (mM) | (mM) | (U/mL) |
| No. 1 | 3 | 0 | 0.0002 |
| No. 2 | 3 | 1.5 | 0.0023 |
| No. 3 | 3 | 3 | 0.0037 |
| No. 4 | 3 | 6 | 0.0040 |
| No. 5 | 3 | 15 | 0.0021 |
| No. 6 | 3 | 30 | 0.0005 |
| No. 7 | 2 | 0 | 0.0003 |
| No. 8 | 2 | 1 | 0.0015 |
| No. 9 | 2 | 2 | 0.0026 |
| No. 10 | 2 | 4 | 0.0031 |
| No. 11 | 2 | 10 | 0.0028 |
| No. 12 | 2 | 20 | 0.0011 |
| No. 13 | 1 | 0 | 0.0003 |
| No. 14 | 1 | 0.5 | 0.0014 |
| No. 15 | 1 | 1 | 0.0022 |
| No. 16 | 1 | 2 | 0.0031 |
| No. 17 | 1 | 5 | 0.0020 |
| No. 18 | 1 | 10 | 0.0014 |
| No. 19 | 1 | 20 | 0.0002 |
| No. 20 | 1 | 30 | 0.0001 |
| No. 21 | 0 | 0 | 0.0000 |

4-2. Study of Additive (Sucrose Vs. Glycerol)

The refolding treatment was performed by the method described in the above “4-1. Study of additive (oxidized glutathione, reduced glutathione)” except that any one of the following (1) and (2) was used as a refolding solution, and the enzyme activity was measured.
(1) 20 mM potassium phosphate buffer (pH 8) to which 1 M sucrose, 3 mM GSSG, 6 mM GSH, and 0.014 w/v % Tween 80 were added
(2) 20 mM potassium phosphate buffer (pH 8) to which 30 w/v % glycerol, 3 mM GSSG, 6 mM GSH, and 0.014 w/v % Tween 80 were added

The results are shown in Table 12. From these results, it was found that the activity is improved about twice by changing the main component of the additive of the refolding solution from sucrose to glycerol.

TABLE 16
(Table 12)

| Additive | Capacity activity (U/mL) |
|---|---|
| 1M Sucrose | 0.0041 |
| 30% Glycerol | 0.0081 |

### 4-3. Study on Influence of Glycerol Concentration in Refolding Solution

The insoluble protein was diluted with a potassium phosphate buffer to obtain a solution by the method described in the above “4-1. Study of additive (oxidized glutathione, reduced glutathione)”. However, the protein concentration after dilution was set to about 0.8 mg/mL. Thereafter, the solution was diluted 50 times with a refolding solution (at the start of refolding: 16 mg/L protein). The composition of the refolding solution was obtained by adding glycerol, 3 mM reduced glutathione (GSSG), 6 mM oxidized glutathione (GSH), and 0.014 w/v % Tween 80 to a 20 mM potassium phosphate buffer (pH 8). At this time, the concentration of glycerol was changed in a range of 0 to 80 w/v % (see Table 13 below).

TABLE 17
(Table 13)

| [symbol] | Glycerol conc. (%) | Capacity activity (U/mL) |
|---|---|---|
| \textcircled{1} | 0 | 0.0001 |
| \textcircled{2} | 10 | 0.0006 |
| \textcircled{3} | 20 | 0.0052 |
| \textcircled{4} | 30 | 0.0084 |
| \textcircled{5} | 40 | 0.0099 |
| \textcircled{6} | 50 | 0.0075 |
| \textcircled{7} | 60 | 0.0075 |
| \textcircled{8} | 70 | 0.0053 |
| \textcircled{9} | 80 | 0.0026 |

[symbol]

indicates data missing or illegible when filed

The results are shown in Table 13. From these results, it was found that the highest enzyme activity is achieved when the glycerol concentration is 40 w/v %.

### 4-4. Comparative Study of Tween 20, 40, 60, and 80 as Additives to Refolding Solution

In addition to the surfactant (Tween 80) in the refolding solution used in the above study, each of Tween 20, 40, and 60, which are Tween-based nonionic surfactants, was used and refolding was examined (the structures of the nonionic surfactants are as follows).

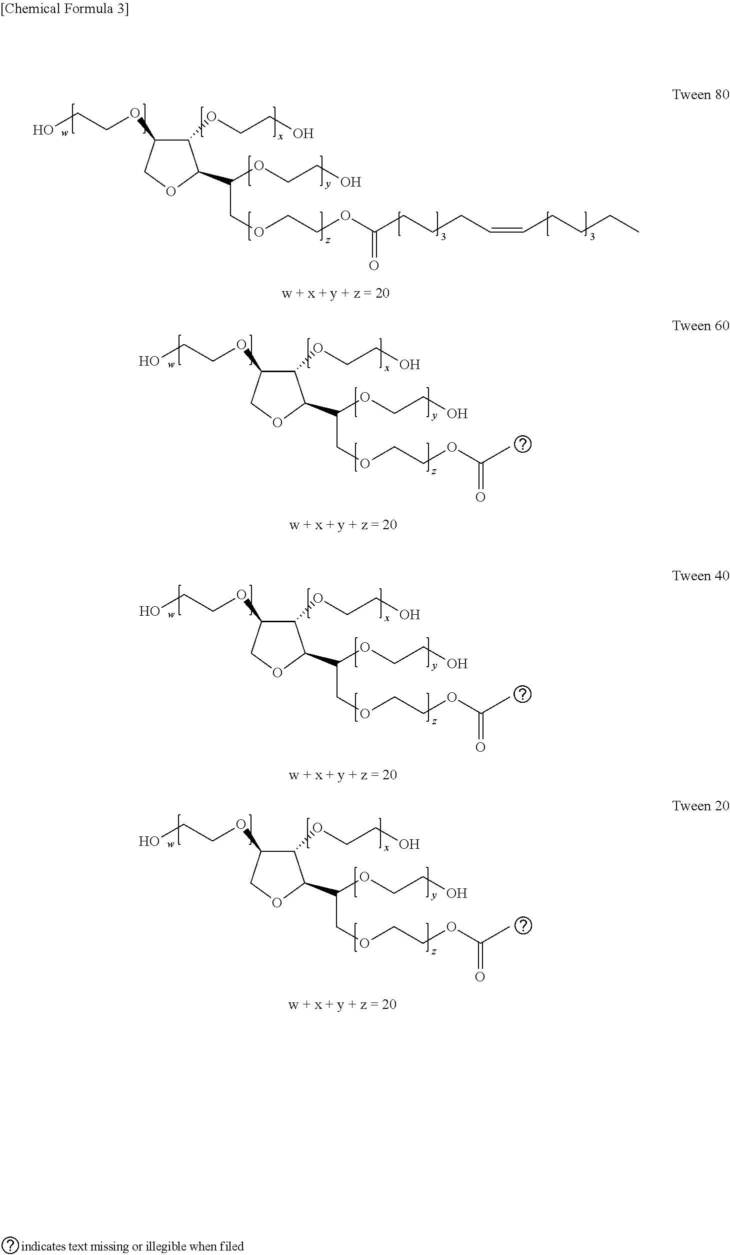

Specifically, first, the insoluble protein was diluted with a potassium phosphate buffer to obtain a solution by the method described in the above “4-1. Study of additive (oxidized glutathione, reduced glutathione)”. Here, the protein concentration after dilution was set to 1.0 mg/mL. Thereafter, the solution was diluted 50 times with a refolding solution (at the start of refolding: 20 mg/L protein). As the refolding solution, a solution obtained by adding 0.25 w/v % or 0.5 w/v % Tween 80, 20, 40, or 60 using, as a base, a 20 mM potassium phosphate buffer (pH 8) to which 40 w/v % glycerol, 3 mM GSSG, and 6 mM GSH were added.

The results are shown in Table 14.

TABLE 14

| | Tween item | | | | | | | | | |
|---|---|---|---|---|---|---|---|---|---|---|
| | Tween 80 (Wako) | | Tween 80 (TCI) | | Tween 60 (TCI) | | Tween 40 (TCI) | | Tween 20 (TCI) | |
| | Tween addition amount (w/v %) | | | | | | | | | |
| | 0.50% | 0.25% | 0.50% | 0.25% | 0.50% | 0.25% | 0.50% | 0.25% | 0.50% | 0.25% |
| After 24 h, capacity | 0.017269 | 0.016754 | 0.01531 | 0.016024 | 0.017978 | 0.016938 | 0.017675 | 0.018617 | 0.014405 | 0.013551 |
| activity (U/mL) | | | | | | | | | | |
| After 46 h, | 0.020358 | 0.020164 | 0.018071 | 0.019206 | 0.022743 | 0.021354 | 0.021769 | 0.023678 | 0.017067 | 0.015916 |
| capacity activity (U/mL) | | | | | | | | | | |
| After 167 h, | 0.021814 | 0.022048 | 0.019606 | 0.02116 | 0.026105 | 0.023742 | 0.024544 | 0.026529 | 0.019227 | 0.017419 |
| capacity activity (U/mL) | | | | | | | | | | |

From these results, better results were obtained in the case of using Tween 60 or Tween 40 as compared with Tween 80.

### 4-5. Evaluation of Influence of Concentration of Surfactant Tween 40 on Refolding

Based on the results of the above 4-4., the influence when Tween 40 was used as a surfactant in the refolding solution and the concentration thereof was changed was examined.

Specifically, first, the insoluble protein was diluted with a potassium phosphate buffer to obtain a solution by the method described in the above “4-1. Study of additive (oxidized glutathione, reduced glutathione)”. Here, the protein concentration after dilution was set to 1.0 mg/mL. Thereafter, the solution was diluted 50 times with a refolding solution (at the start of refolding: 20 mg/L protein). As the refolding solution, a solution obtained by adding 0.25 w/v& Tween 80 or 0.1 to 0.4 w/v % Tween 40 using, as a base, a 20 mM potassium phosphate buffer (pH 8) to which 40 w/v& glycerol, 3 mM GSSG, and 6 mM GSH were added. The results are shown in Table 15.

TABLE 19
(Table 15)

| | Addition concentration | Specific activity |
|---|---|---|
| Item | (w/v %) | (U/mg) |
| Tween 80 (Wako) | 0.25 | 0.0219793 |
| Tween 40 (TCI) | 0.10 | 0.0251698 |
| | 0.20 | 0.0270317 |
| | 0.30 | 0.0272919 |
| | 0.40 | 0.0261481 |

From these results, the best results were obtained when Tween 40 at a concentration of 0.3 w/v % was used.

### 4-6. Evaluation of Influence of Redox Agent During Refolding

The influence when the type and concentration of the redox agent in the refolding solution was examined.

Specifically, first, the insoluble protein was diluted with a potassium phosphate buffer to obtain a solution by the method described in the above “4-1. Study of additive (oxidized glutathione, reduced glutathione)”. Here, the protein concentration after dilution was set to 1.0 mg/mL. Thereafter, the solution was diluted 50 times with a refolding solution (at the start of refolding: 20 mg/L protein). As the refolding solution, those obtained by using, as a base, a 20 mM potassium phosphate buffer (pH 8) to which 40 w/v % glycerol and 0.25 w/v % Tween 80 were added and using the type and concentration of redox agent shown in Table 16 below were adopted. The results are shown in Table 16.

TABLE 20
(Table 16)

| | | Conc. | Capacity activity |
|---|---|---|---|
| No. | Reagent | (mM) | (U/mL) |
| \textcircled{1} | GSH | 6 | 0.017 |
| | GSSG | 3 | |
| \textcircled{2} | GSH | 4 | 0.017 |
| | GSSG | 2 | |
| \textcircled{3} | GSH | 2 | 0.017 |
| | GSSG | 1 | |
| \textcircled{4} | Cys | 2 | 0.019 |
| | Cys-Cys | 1 | |
| \textcircled{5} | Cys | 1 | 0.005 |
| | Cys-Cys | 0.5 | |
| \textcircled{6} | GSH | 2 | 0.016 |
| | Cys-Cys | 1 | |

From these results, the condition of “1 mM cystine (Cys-Cys) and 2 mM cysteine (Cys)” showed a better result than the condition of “3 mM GSSG and 6 mM GSH” which was considered to be the optimum condition in the above examination.

### 4-7. Optimization Study of Cystine/Cysteine Concentration During Refolding

Based on the results of the above 4-6., those obtained by using, as a base, a 20 mM potassium phosphate buffer (pH 8) to which 40 w/v % glycerol, 0.25 w/v % Tween 80, and 0.3 w/v Tween 40 were added and adding cystine (Cys-Cys) and cysteine (Cys) at a concentration combination shown in Table 17 below were used as a refolding solution, and refolding was examined. The results are shown in Table 17.

TABLE 21
(Table 17)

| | | |
|---|---|---|
| Cysteine | Cystine | After refolding treatment (h) |

| (mM) | (mM) | 24.5 | 49 | 167 |
|---|---|---|---|---|
| 0.5 | 1 | 0.000 | 0.000 | 0.000 |
| 1 | 1 | 0.001 | 0.001 | 0.001 |
| 1.5 | 1 | 0.003 | 0.004 | 0.003 |
| 2 | 1 | 0.005 | 0.008 | 0.009 |
| 3 | 1 | 0.010 | 0.013 | 0.015 |
| 4 | 1 | 0.012 | 0.015 | 0.018 |
| 5 | 1 | 0.014 | 0.017 | 0.019 |
| 1 | 2 | 0.000 | 0.000 | 0.000 |
| 2 | 2 | 0.004 | 0.006 | 0.008 |
| 3 | 2 | 0.008 | 0.012 | 0.016 |
| 4 | 2 | 0.011 | 0.015 | 0.019 |
| 6 | 2 | 0.015 | 0.017 | 0.020 |
| 8 | 2 | 0.015 | 0.017 | 0.019 |
| 10 | 2 | 0.015 | 0.017 | 0.019 |
| 1.5 | 3 | 0.002 | 0.003 | 0.001 |
| 3 | 3 | 0.008 | 0.013 | 0.015 |
| 4.5 | 3 | 0.013 | 0.017 | 0.019 |
| 6 | 3 | 0.015 | 0.018 | 0.020 |
| 9 | 3 | 0.015 | 0.017 | 0.019 |
| 12 | 3 | 0.015 | 0.017 | 0.018 |

From these results, it was found that the maximum capacity activity is achieved when the refolding treatment is performed using a refolding solution to which 2 mM cystine (Cys-Cys) and 6 mM cysteine (Cys) are added as redox agents.

The present application is based on Japanese Patent Application No. 2022-013052 filed on Jan. 31, 2022, the disclosure content of which is incorporated herein by reference in its entirety.

1. A protein,
(a) comprising an amino acid sequence set forth in SEQ ID NO: 1 or 2 or an amino acid sequence having identity of 90% or more therewith,
(b) being added with no sugar chain, and
(c) having glucocerebrosidase activity.

2. The protein according to claim 1, which comprises at least one of amino acid substitutions below in the amino acid sequence of SEQ ID NO: 1 or 2:
(1) a substitution of an amino acid at a position corresponding to position 26 of SEQ ID NO: 1 or 2 with leucine (F26L);
(2) a substitution of an amino acid at a position corresponding to position 26 of SEQ ID NO: 1 or 2 with isoleucine (F26I);
(3) a substitution of an amino acid at a position corresponding to position 126 of SEQ ID NO: 1 or 2 with threonine (C126T);
(4) a substitution of an amino acid at a position corresponding to position 126 of SEQ ID NO: 1 or 2 with serine (C126S) and a substitution of an amino acid at a position corresponding to position 342 of SEQ ID NO: 1 or 2 with serine (C342S);
(5) a substitution of an amino acid at a position corresponding to position 57 of SEQ ID NO: 1 or 2 with cysteine (Q57C);
(6) a substitution of an amino acid at a position corresponding to position 60 of SEQ ID NO: 1 or 2 with cysteine (H60C);
(7) a substitution of an amino acid at a position corresponding to position 63 of SEQ ID NO: 1 or 2 with cysteine (T63C);
(8) a substitution of an amino acid at a position corresponding to position 143 of SEQ ID NO: 1 or 2 with cysteine (Q143C);
(9) a substitution of an amino acid at a position corresponding to position 145 of SEQ ID NO: 1 or 2 with cysteine (H145C);
(10) a substitution of an amino acid at a position corresponding to position 224 of SEQ ID NO: 1 or 2 with cysteine (K224C); and
(11) a substitution of an amino acid at a position corresponding to position 321 of SEQ ID NO: 1 or 2 with cysteine (K321C).

3. The protein according to claim 1, which comprises at least one of amino acid substitutions below in the amino acid sequence of SEQ ID NO: 1 or 2:
(12) a substitution of an amino acid at a position corresponding to position 248 of SEQ ID NO: 1 or 2 with serine (C248S) and a substitution of an amino acid at a position corresponding to position 342 of SEQ ID NO: 1 or 2 with serine (C342S);
(13) a substitution of an amino acid at a position corresponding to position 126 of SEQ ID NO: 1 or 2 with threonine (C126T) and a substitution of an amino acid at a position corresponding to position 342 of SEQ ID NO: 1 or 2 with serine (C342S);
(14) a substitution of an amino acid at a position corresponding to position 126 of SEQ ID NO: 1 or 2 with serine (C126S), a substitution of an amino acid at a position corresponding to position 248 of SEQ ID NO: 1 or 2 with serine (C248S), and a substitution of an amino acid at a position corresponding to position 342 of SEQ ID NO: 1 or 2 with serine (C342S); and
(15) a substitution of an amino acid at a position corresponding to position 126 of SEQ ID NO: 1 or 2 with threonine (C126T), a substitution of an amino acid at a position corresponding to position 248 of SEQ ID NO: 1 or 2 with serine (C248S), and a substitution of an amino acid at a position corresponding to position 342 of SEQ ID NO: 1 or 2 with serine (C342S).

4. The protein according to claim 1, which comprises at least one of amino acid substitutions below in the amino acid sequence of SEQ ID NO: 1 or 2:
(16) a substitution of an amino acid at a position corresponding to position 61 of SEQ ID NO: 1 or 2 with cysteine (T61C);
(17) a substitution of an amino acid at a position corresponding to position 98 of SEQ ID NO: 1 or 2 with cysteine (P98C);
(18) a substitution of an amino acid at a position corresponding to position 143 of SEQ ID NO: 1 or 2 with cysteine (Q143C);
(19) a substitution of an amino acid at a position corresponding to position 224 of SEQ ID NO: 1 or 2 with cysteine (K224C);
(20) a substitution of an amino acid at a position corresponding to position 321 of SEQ ID NO: 1 or 2 with cysteine (K321C); and
(21) a substitution of an amino acid at a position corresponding to position 407 of SEQ ID NO: 1 or 2 with cysteine (T407C).

5. The protein according to claim 1, which is produced by a prokaryote.

6. A method for producing a protein having glucocerebrosidase activity, the method comprising subjecting the protein according to claim 1, which is not folded, to a folding treatment.

7. A method for producing a protein having glucocerebrosidase activity, comprising:
introducing a vector containing a nucleic acid encoding the protein according to claim 1 into a prokaryote to cause the prokaryote to produce the protein; and
subjecting the protein produced by the prokaryote, which is collected, to a folding treatment.

8. A protein having glucocerebrosidase activity,
which is produced by refolding the protein according to claim 5.


SEQUENCE LISTING (135 sequences)

SEQ ID NO: 1 - protein, 497 aa, synthetic construct [sequence omitted]

SEQ ID NO: 2 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 3 - protein, 497 aa, synthetic construct
  REGION 1..497: note = protein having glucocerebrosidase activity
ARPCIPKSFGYSSVVCVCNATYCDSLDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 4 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASSDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 5 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 6 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEAGVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 7 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 8 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEATVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 9 - protein, 497 aa, synthetic construct
  REGION 1..497: note = protein having glucocerebrosidase activity
ARPCIPKSFGYSSVVCVCNATYCDSIDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 10 - protein, 497 aa, synthetic construct
  REGION 1..497: note = protein having glucocerebrosidase activity
ARPCIPKSFGYSSVVCVCNATYCDSLDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 11 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASSDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 12 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 13 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASSDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 14 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 15 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFCLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 16 - protein, 497 aa, synthetic construct
  REGION 1..497: note = protein having glucocerebrosidase activity
ARPCIPKSFGYSSVVCVCNATYCDSLDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 17 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASSDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 18 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 19 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPCQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 20 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPICANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 21 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQCNHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 22 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQACHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 23 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANCTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 24 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 25 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTCTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 26 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGCGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 27 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTCLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 28 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSCPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 29 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDCQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 30 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 31 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQCHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 32 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLCNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 33 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHCFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 34 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNCSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 35 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFCLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 36 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRCVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 37 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHCLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 38 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDCEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 39 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPACATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 40 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKCWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 41 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDICKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 42 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQCNDLDAVALMHPDGSAVVVVLNRSSKDVPLTIKDPAVGFLETISPGYSIHTYLWHRQ

SEQ ID NO: 43 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 44 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQCVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 45 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPCWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 46 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWACVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 47 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLCDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 48 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPCTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 49 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSCDVPLTIKDPAVGFLETISPGYSIHTYLWHRQ

SEQ ID NO: 50 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 51 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 52 - DNA, 21 bp, synthetic construct
  misc_feature 1..21: note = primer
  source 1..21: mol_type = other DNA
ctggatccgcccacttttccc

SEQ ID NO: 53 - DNA, 18 bp, synthetic construct
  misc_feature 1..18: note = primer
  source 1..18: mol_type = other DNA
cgaatcgcagtacgtggc

SEQ ID NO: 54 - DNA, 26 bp, synthetic construct
  misc_feature 1..26: note = primer
  source 1..26: mol_type = other DNA
agcgacttttcgattcgcacctatac

SEQ ID NO: 55 - DNA, 18 bp, synthetic construct
  misc_feature 1..18: note = primer
  source 1..18: mol_type = other DNA
cgaggccattggaacgcg

SEQ ID NO: 56 - DNA, 23 bp, synthetic construct
  misc_feature 1..23: note = primer
  source 1..23: mol_type = other DNA
gacttttcgattcgcacctatac

SEQ ID NO: 57 - DNA, 21 bp, synthetic construct
  misc_feature 1..21: note = primer
  source 1..21: mol_type = other DNA
ggtcgaggccattggaacgcg

SEQ ID NO: 58 - DNA, 21 bp, synthetic construct
  misc_feature 1..21: note = primer
  source 1..21: mol_type = other DNA
gtggggagcaagttttgggag

SEQ ID NO: 59 - DNA, 25 bp, synthetic construct
  misc_feature 1..25: note = primer
  source 1..25: mol_type = other DNA
gcctgcttcgctagcaaagagcatc

SEQ ID NO: 60 - DNA, 26 bp, synthetic construct
  misc_feature 1..26: note = primer
  source 1..26: mol_type = other DNA
aagcaagcgtggggagcaagttttgg

SEQ ID NO: 61 - DNA, 27 bp, synthetic construct
  misc_feature 1..27: note = primer
  source 1..27: mol_type = other DNA
cccacgcttgcttcgctagcaaagagc

SEQ ID NO: 62 - DNA, 26 bp, synthetic construct
  misc_feature 1..26: note = primer
  source 1..26: mol_type = other DNA
aagcaaccgtggggagcaagttttgg

SEQ ID NO: 63 - DNA, 27 bp, synthetic construct
  misc_feature 1..27: note = primer
  source 1..27: mol_type = other DNA
cccacggttgcttcgctagcaaagagc

SEQ ID NO: 64 - DNA, 21 bp, synthetic construct
  misc_feature 1..21: note = primer
  source 1..21: mol_type = other DNA
attgatccgcccacttttccc

SEQ ID NO: 65 - DNA, 18 bp, synthetic construct
  misc_feature 1..18: note = primer
  source 1..18: mol_type = other DNA
cgaatcgcagtacgtggc

SEQ ID NO: 66 - DNA, 22 bp, synthetic construct
  misc_feature 1..22: note = primer
  source 1..22: mol_type = other DNA
agcctgggttttacgccggaac

SEQ ID NO: 67 - DNA, 21 bp, synthetic construct
  misc_feature 1..21: note = primer
  source 1..21: mol_type = other DNA
ttgaaacggataacccgagag

SEQ ID NO: 68 - DNA, 25 bp, synthetic construct
  misc_feature 1..25: note = primer
  source 1..25: mol_type = other DNA
tgcttaccggaagaggatacgaaac

SEQ ID NO: 69 - DNA, 24 bp, synthetic construct
  misc_feature 1..24: note = primer
  source 1..24: mol_type = other DNA
gaaattgtgcagctgaaagtcatc

SEQ ID NO: 70 - DNA, 23 bp, synthetic construct
  misc_feature 1..23: note = primer
  source 1..23: mol_type = other DNA
tgcggtactgggctgttgcttac

SEQ ID NO: 71 - DNA, 20 bp, synthetic construct
  misc_feature 1..20: note = primer
  source 1..20: mol_type = other DNA
gtgattggcctgaatagggc

SEQ ID NO: 72 - DNA, 20 bp, synthetic construct
  misc_feature 1..20: note = primer
  source 1..20: mol_type = other DNA
caggccaatcacaccggtac

SEQ ID NO: 73 - DNA, 22 bp, synthetic construct
  misc_feature 1..22: note = primer
  source 1..22: mol_type = other DNA
gcaagggcccatagacagttcc

SEQ ID NO: 74 - DNA, 19 bp, synthetic construct
  misc_feature 1..19: note = primer
  source 1..19: mol_type = other DNA
gccaatcacaccggtactg

SEQ ID NO: 75 - DNA, 24 bp, synthetic construct
  misc_feature 1..24: note = primer
  source 1..24: mol_type = other DNA
gcaaatagggcccatagacagttc

SEQ ID NO: 76 - DNA, 18 bp, synthetic construct
  misc_feature 1..18: note = primer
  source 1..18: mol_type = other DNA
aatcacaccggtactggg

SEQ ID NO: 77 - DNA, 22 bp, synthetic construct
  misc_feature 1..22: note = primer
  source 1..22: mol_type = other DNA
gcactgaatagggcccatagac

SEQ ID NO: 78 - DNA, 26 bp, synthetic construct
  misc_feature 1..26: note = primer
  source 1..26: mol_type = other DNA
aggcctgccacaccggtactgggctg

SEQ ID NO: 79 - DNA, 26 bp, synthetic construct
  misc_feature 1..26: note = primer
  source 1..26: mol_type = other DNA
gagaagcagtgcagctgaaagtcatc

SEQ ID NO: 80 - DNA, 18 bp, synthetic construct
  misc_feature 1..18: note = primer
  source 1..18: mol_type = other DNA
accggtactgggctgttg

SEQ ID NO: 81 - DNA, 21 bp, synthetic construct
  misc_feature 1..21: note = primer
  source 1..21: mol_type = other DNA
gcaattggcctgaatagggcc

SEQ ID NO: 82 - DNA, 23 bp, synthetic construct
  misc_feature 1..23: note = primer
  source 1..23: mol_type = other DNA
tgcggtactgggctgttgcttac

SEQ ID NO: 83 - DNA, 20 bp, synthetic construct
  misc_feature 1..20: note = primer
  source 1..20: mol_type = other DNA
gtgattggcctgaatagggc

SEQ ID NO: 84 - DNA, 18 bp, synthetic construct
  misc_feature 1..18: note = primer
  source 1..18: mol_type = other DNA
actgggctgttgcttacc

SEQ ID NO: 85 - DNA, 23 bp, synthetic construct
  misc_feature 1..23: note = primer
  source 1..23: mol_type = other DNA
gcaggtgtgattggcctgaatag

SEQ ID NO: 86 - DNA, 18 bp, synthetic construct
  misc_feature 1..18: note = primer
  source 1..18: mol_type = other DNA
gggctgttgcttaccctc

SEQ ID NO: 87 - DNA, 21 bp, synthetic construct
  misc_feature 1..21: note = primer
  source 1..21: mol_type = other DNA
gcaaccggtgtgattggcctg

SEQ ID NO: 88 - DNA, 19 bp, synthetic construct
  misc_feature 1..19: note = primer
  source 1..19: mol_type = other DNA
ctgttgcttaccctccaac

SEQ ID NO: 89 - DNA, 21 bp, synthetic construct
  misc_feature 1..21: note = primer
  source 1..21: mol_type = other DNA
gcaagtaccggtgtgattggc

SEQ ID NO: 90 - DNA, 22 bp, synthetic construct
  misc_feature 1..22: note = primer
  source 1..22: mol_type = other DNA
tgcctgggttttacgccggaac

SEQ ID NO: 91 - DNA, 21 bp, synthetic construct
  misc_feature 1..21: note = primer
  source 1..21: mol_type = other DNA
ttgaaacggataacccgagag

SEQ ID NO: 92 - DNA, 22 bp, synthetic construct
  misc_feature 1..22: note = primer
  source 1..22: mol_type = other DNA
cagctgcacaatttctcgttac

SEQ ID NO: 93 - DNA, 21 bp, synthetic construct
  misc_feature 1..21: note = primer
  source 1..21: mol_type = other DNA
gcagtcatccggcgtatccgc

SEQ ID NO: 94 - DNA, 20 bp, synthetic construct
  misc_feature 1..20: note = primer
  source 1..20: mol_type = other DNA
ctgcacaatttctcgttacc

SEQ ID NO: 95 - DNA, 21 bp, synthetic construct
  misc_feature 1..21: note = primer
  source 1..21: mol_type = other DNA
gcaaaagtcatccggcgtatc

SEQ ID NO: 96 - DNA, 22 bp, synthetic construct
  misc_feature 1..22: note = primer
  source 1..22: mol_type = other DNA
cacaatttctcgttaccggaag

SEQ ID NO: 97 - DNA, 24 bp, synthetic construct
  misc_feature 1..24: note = primer
  source 1..24: mol_type = other DNA
gcactgaaagtcatccggcgtatc

SEQ ID NO: 98 - DNA, 22 bp, synthetic construct
  misc_feature 1..22: note = primer
  source 1..22: mol_type = other DNA
aatttctcgttaccggaagagg

SEQ ID NO: 99 - DNA, 22 bp, synthetic construct
  misc_feature 1..22: note = primer
  source 1..22: mol_type = other DNA
gcacagctgaaagtcatccggc

SEQ ID NO: 100 - DNA, 26 bp, synthetic construct
  misc_feature 1..26: note = primer
  source 1..26: mol_type = other DNA
tgcactgcttctcgttaccggaagag

SEQ ID NO: 101 - DNA, 28 bp, synthetic construct
  misc_feature 1..28: note = primer
  source 1..28: mol_type = other DNA
gtgtggcaggcctgaatagggcccatag

SEQ ID NO: 102 - DNA, 21 bp, synthetic construct
  misc_feature 1..21: note = primer
  source 1..21: mol_type = other DNA
tcgttaccggaagaggatacg

SEQ ID NO: 103 - DNA, 24 bp, synthetic construct
  misc_feature 1..24: note = primer
  source 1..24: mol_type = other DNA
gcaattgtgcagctgaaagtcatc

SEQ ID NO: 104 - DNA, 25 bp, synthetic construct
  misc_feature 1..25: note = primer
  source 1..25: mol_type = other DNA
tgcttaccggaagaggatacgaaac

SEQ ID NO: 105 - DNA, 24 bp, synthetic construct
  misc_feature 1..24: note = primer
  source 1..24: mol_type = other DNA
gaaattgtgcagctgaaagtcatc

SEQ ID NO: 106 - DNA, 19 bp, synthetic construct
  misc_feature 1..19: note = primer
  source 1..19: mol_type = other DNA
gtgtccctgctggcttcac

SEQ ID NO: 107 - DNA, 22 bp, synthetic construct
  misc_feature 1..22: note = primer
  source 1..22: mol_type = other DNA
gcaacgctgtgccaattgtaac

SEQ ID NO: 108 - DNA, 18 bp, synthetic construct
  misc_feature 1..18: note = primer
  source 1..18: mol_type = other DNA
ctgcagttttgggccgtg

SEQ ID NO: 109 - DNA, 21 bp, synthetic construct
  misc_feature 1..21: note = primer
  source 1..21: mol_type = other DNA
gcaatgctcagcatacgcatc

SEQ ID NO: 110 - DNA, 18 bp, synthetic construct
  misc_feature 1..18: note = primer
  source 1..18: mol_type = other DNA
gaagctgcgaaatatgtg

SEQ ID NO: 111 - DNA, 22 bp, synthetic construct
  misc_feature 1..22: note = primer
  source 1..22: mol_type = other DNA
gcaatcggttaacaccactttg

SEQ ID NO: 112 - DNA, 19 bp, synthetic construct
  misc_feature 1..19: note = primer
  source 1..19: mol_type = other DNA
gccaccttgggagaaactc

SEQ ID NO: 113 - DNA, 21 bp, synthetic construct
  misc_feature 1..21: note = primer
  source 1..21: mol_type = other DNA
gcaggcaggcgccagaaaatc

SEQ ID NO: 114 - DNA, 18 bp, synthetic construct
  misc_feature 1..18: note = primer
  source 1..18: mol_type = other DNA
ctgggagcaaagcgtccg

SEQ ID NO: 115 - DNA, 19 bp, synthetic construct
  misc_feature 1..19: note = primer
  source 1..19: mol_type = other DNA
cacttgctccccacgcttg

SEQ ID NO: 116 - DNA, 22 bp, synthetic construct
  misc_feature 1..22: note = primer
  source 1..22: mol_type = other DNA
aaggatacgttctacaaacagc

SEQ ID NO: 117 - DNA, 27 bp, synthetic construct
  misc_feature 1..27: note = primer
  source 1..27: mol_type = other DNA
gcagatatcgacaatgatcggagaatc

SEQ ID NO: 118 - DNA, 20 bp, synthetic construct
  misc_feature 1..20: note = primer
  source 1..20: mol_type = other DNA
aacgacctagatgcagtagc

SEQ ID NO: 119 - DNA, 21 bp, synthetic construct
  misc_feature 1..21: note = primer
  source 1..21: mol_type = other DNA
gcactgggaggctaccaggcc

SEQ ID NO: 120 - DNA, 18 bp, synthetic construct
  misc_feature 1..18: note = primer
  source 1..18: mol_type = other DNA
gtgaaaggctttggcggc

SEQ ID NO: 121 - DNA, 25 bp, synthetic construct
  misc_feature 1..25: note = primer
  source 1..25: mol_type = other DNA
gcactgaaatttctgctccggttgg

SEQ ID NO: 122 - DNA, 20 bp, synthetic construct
  misc_feature 1..20: note = primer
  source 1..20: mol_type = other DNA
tgggccaaagtggtgttaac

SEQ ID NO: 123 - DNA, 21 bp, synthetic construct
  misc_feature 1..21: note = primer
  source 1..21: mol_type = other DNA
gcacggaaggagcaaccgttg

SEQ ID NO: 124 - DNA, 22 bp, synthetic construct
  misc_feature 1..22: note = primer
  source 1..22: mol_type = other DNA
gatccagaagctgcgaaatatg

SEQ ID NO: 125 - DNA, 23 bp, synthetic construct
  misc_feature 1..23: note = primer
  source 1..23: mol_type = other DNA
gcataacaccactttggcccaat

SEQ ID NO: 126 - DNA, 20 bp, synthetic construct
  misc_feature 1..20: note = primer
  source 1..20: mol_type = other DNA
acgatgctctttgctagcga

SEQ ID NO: 127 - DNA, 24 bp, synthetic construct
  misc_feature 1..24: note = primer
  source 1..24: mol_type = other DNA
gcacggaaagaggcgatgagtttc

SEQ ID NO: 128 - DNA, 18 bp, synthetic construct
  misc_feature 1..18: note = primer
  source 1..18: mol_type = other DNA
gacgtaccgctgaccatc

SEQ ID NO: 129 - DNA, 24 bp, synthetic construct
  misc_feature 1..24: note = primer
  source 1..24: mol_type = other DNA
gcatgaactgcggtttaaaacaac

SEQ ID NO: 130 - DNA, 26 bp, synthetic construct
  misc_feature 1..26: note = primer
  source 1..26: mol_type = other DNA
cagtaaggatccgaattcgagctccg

SEQ ID NO: 131 - DNA, 59 bp, synthetic construct
  misc_feature 1..59: note = primer
  source 1..59: mol_type = other DNA
tgatgatgatggcccatggtatatctccttcttaaagttaaacaaaattatttctagag

SEQ ID NO: 132 - DNA, 81 bp, synthetic construct
  misc_feature 1..81: note = primer
  source 1..81: mol_type = other DNA
gggccatcatcatcatcatcatcatcatcatcacagcagcggccatatcgacgacgacgacaaggcgcgcccatgtatccc

SEQ ID NO: 133 - DNA, 34 bp, synthetic construct
  misc_feature 1..34: note = primer
  source 1..34: mol_type = other DNA
ttcggatccttactgacgatgccacagataggtg

SEQ ID NO: 134 - DNA, 1494 bp, synthetic construct
  misc_feature 1..1494: note = synthetic polypeptide
  source 1..1494: mol_type = other DNA
gcgcgcccatgtatcccgaaaagcttcggttatagttctgttgtctgtgtctgtaatgccacgtactgcgattcgtttgatccgcccacttttcccgcgttaggtaccttcagccgttatgagagcacacgttcaggacgtcgcatggaactgtctatgggccctattcaggccaatcacaccggtactgggctgttgcttaccctccaaccggagcagaaatttcagaaagtgaaaggctttggcggcgcaatgacggacgcagccgctcttaatattctggcgctgtctccgccggcacaaaacctgttgctgaaatcctacttcagtgaagaaggcataggctacaacatcatacgcgttccaatggcctcgtgcgacttttcgattcgcacctatacgtatgcggatacgccggatgactttcagctgcacaatttctcgttaccggaagaggatacgaaactcaagatcccgttaattcaccgcgcgttacaattggcacagcgtcctgtgtccctgctggcttcaccgtggacatccccgacatggctgaaaaccaatggagctgtcaacggcaaagggtctctgaaaggtcaaccaggcgatatctatcaccagacctgggcgaggtatttcgtgaaattcctggatgcgtatgctgagcataagctgcagttttgggccgtgacagccgaaaacgagccaagtgcaggtttgctctcgggttatccgtttcaatgcctgggttttacgccggaacatcagcgtgacttcattgcgcgtgacttgggccccactctggcgaatagtacccaccacaatgtccggctgctgatgctggacgatcaacggttgctccttccgcattgggccaaagtggtgttaaccgatccagaagctgcgaaatatgtgcatggtatcgcggttcactggtatctggattttctggcgcctgccaaagccaccttgggagaaactcatcgcctctttccgaacacgatgctctttgctagcgaagcatgcgtggggagcaagttttgggagcaaagcgtccgcttaggcagttgggatcgaggcatgcagtacagccatagcatcattaccaacctgctgtaccatgttgttggctggactgactggaacctagcccttaatccggaaggtggtccgaattgggtacgtaactttgtggattctccgatcattgtcgatatcaccaaggatacgttctacaaacagcccatgttctaccatctggggcatttttcgaagttcattcctgaagggtcacagagagttggcctggtagcctcccagaaaaacgacctagatgcagtagcgctgatgcatccagatggcagcgcagtggtggttgttttaaaccgcagttcaaaagacgtaccgctgaccatcaaagaccctgcggtaggtttccttgaaaccattagcccaggatactccattcacacctatctgtggcatcgtcagtaa

SEQ ID NO: 135 - DNA, 1497 bp, synthetic construct
  misc_feature 1..1497: note = synthetic polypeptide
  source 1..1497: mol_type = other DNA
atggcgcgcccatgtatcccgaaaagcttcggttatagttctgttgtctgtgtctgtaatgccacgtactgcgattcgtttgatccgcccacttttcccgcgttaggtaccttcagccgttatgagagcacacgttcaggacgtcgcatggaactgtctatgggccctattcaggccaatcacaccggtactgggctgttgcttaccctccaaccggagcagaaatttcagaaagtgaaaggctttggcggcgcaatgacggacgcagccgctcttaatattctggcgctgtctccgccggcacaaaacctgttgctgaaatcctacttcagtgaagaaggcataggctacaacatcatacgcgttccaatggcctcgtgcgacttttcgattcgcacctatacgtatgcggatacgccggatgactttcagctgcacaatttctcgttaccggaagaggatacgaaactcaagatcccgttaattcaccgcgcgttacaattggcacagcgtcctgtgtccctgctggcttcaccgtggacatccccgacatggctgaaaaccaatggagctgtcaacggcaaagggtctctgaaaggtcaaccaggcgatatctatcaccagacctgggcgaggtatttcgtgaaattcctggatgcgtatgctgagcataagctgcagttttgggccgtgacagccgaaaacgagccaagtgcaggtttgctctcgggttatccgtttcaatgcctgggttttacgccggaacatcagcgtgacttcattgcgcgtgacttgggccccactctggcgaatagtacccaccacaatgtccggctgctgatgctggacgatcaacggttgctccttccgcattgggccaaagtggtgttaaccgatccagaagctgcgaaatatgtgcatggtatcgcggttcactggtatctggattttctggcgcctgccaaagccaccttgggagaaactcatcgcctctttccgaacacgatgctctttgctagcgaagcatgcgtggggagcaagttttgggagcaaagcgtccgcttaggcagttgggatcgaggcatgcagtacagccatagcatcattaccaacctgctgtaccatgttgttggctggactgactggaacctagcccttaatccggaaggtggtccgaattgggtacgtaactttgtggattctccgatcattgtcgatatcaccaaggatacgttctacaaacagcccatgttctaccatctggggcatttttcgaagttcattcctgaagggtcacagagagttggcctggtagcctcccagaaaaacgacctagatgcagtagcgctgatgcatccagatggcagcgcagtggtggttgttttaaaccgcagttcaaaagacgtaccgctgaccatcaaagaccctgcggtaggtttccttgaaaccattagcccaggatactccattcacacctatctgtggcatcgtcagtaa